(19) United States
(12) Patent Application Publication
(10) Pub. No.: US 2024/0341768 A1
(43) Pub. Date: Oct. 17, 2024

(54) VALVES TO CONTROL WIRE MOVEMENT

(71) Applicants: DePuy Synthes Products, Inc., Raynham, MA (US); Neuravi Limited, Gatway (IE)

(72) Inventors: Anushree DWIVEDI, Galway (IE); Thomas GALLERANI, Miami, FL (US); Melissa CLINGER, Raynham, MA (US); Chelsea VIJIL, Miami, FL (US)

(73) Assignees: DePuy Synthes Products, Inc., Raynham, MA (US); Neuravi Limited, Galway (IE)

(21) Appl. No.: 18/135,569

(22) Filed: Apr. 17, 2023

(51) Int. Cl. A61B 17/12; A61F 2/01

(52) U.S. Cl. CPC A61B 17/12109; A61B 17/1214; A61F 2/011

(57) ABSTRACT

An apparatus for controlling movement of a wire configured to deploy an implant is presented herein. The apparatus can include a wire including a bump and a valve including a friction element configured to apply a friction force to the wire. The friction force can vary based on one or more of: a speed at which the wire is pulled through the friction element, a direction in which the wire is pulled through the friction element, and a contact between the bump and the friction element. The wire can be configured to deploy an implant upon a proximal retraction of the wire.

### FIELD

The present invention generally relates to implantable medical devices, and more particularly to implants to induce venous stasis.

### BACKGROUND

Aneurysms can be intravascularly treated by delivering a treatment device to the aneurysm to fill the sac of the aneurysm with embolic material and/or block the neck of the aneurysm to inhibit blood flow into the aneurysm. When filling the aneurysm sac, the embolic material can promote blood clotting to create a thrombotic mass within the aneurysm. When treating the aneurysm neck without substantially filling the aneurysm sac, blood flow into the neck of the aneurysm can be inhibited to induce venous stasis in the aneurysm and facilitate natural formation of a thrombotic mass within the aneurysm.

In some current treatments, embolic coils are used to either fill the aneurysm sac or treat the entrance of the aneurysm neck. Embolic coils may be released or deployed by mechanical means with a delivery catheter and wire system. Many such catheter-based delivery systems include a wire for retention of the implant in the catheter until the time for release of the device. These systems are then actuated by retracting or pulling the wire relative to the catheter. Such a wire is sometimes referred to as a “pull wire”.

One issue with current catheter-based delivery systems is premature detachment of the implantable device. Premature detachment occurs when the implant is detached from the delivery system before reaching the treatment site. This may occur due to the tortuosity experienced by the delivery system as it passes through the vasculature of the patient, which can cause an increase in friction between the “pull wire” and the delivery system causing the pull wire to move proximally while the delivery system is moving distally.

Accordingly, there is a need for an improved implant delivery system that prevents premature detachment of the implant as it is delivered through tortuous vasculature. This disclosure is directed to this and other considerations.

### SUMMARY

In some examples, an apparatus for controlling movement of a wire configured to deploy an implant is disclosed. The apparatus can include a wire including a bump and a valve including a friction element configured to apply a friction force to the wire. The friction force can vary based on one or more of: a speed at which the wire is pulled through the friction element, a direction in which the wire is pulled through the friction element, and a contact between the bump and the friction element. The wire can be configured to deploy an implant upon a proximal retraction of the wire.

In some examples, a system for delivering an implant is disclosed. The system can include a delivery tube, a wire disposed in the delivery tube and configured to deploy an implant upon a proximal retraction of the wire, and a valve including a friction element configured to apply a friction force to the wire. The friction force can vary based on one or more of a speed at which the wire is pulled through the friction element, a direction in which the wire is pulled through the friction element, and a contact between the wire and the friction element. The delivery tube can include a distal spring portion, an intermediate flexible portion, and a proximal portion making up a majority of a length of the delivery tube.

In some examples, a method of deploying an implant is disclosed. The method can include positioning a distal end of a delivery tube while hindering a premature deployment of an implant and deploying the implant. Deploying the implant can include retracting a wire proximally by pulling the wire with a force greater than a minimum deployment force, the premature deployment of the implant being caused by proximal movement of a distal portion of the wire in relation to a delivery tube.

Other aspects and features of the present disclosure will become apparent to those skilled in the pertinent art, upon reviewing the following detailed description in conjunction with the accompanying figures.

### BRIEF DESCRIPTION OF THE DRAWINGS

The above and further aspects of this disclosure are further discussed with the following description of the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation. It is expected that those of skill in the art can conceive of and combining elements from multiple figures to better suit the needs of the user.

FIG. 1A illustrates a side cutaway view of an apparatus for controlling movement of a wire, according to this disclosure;

FIG. 1B illustrates an isometric cutaway view of an apparatus for controlling movement of a wire, according to this disclosure;

FIG. 2A is a front view of a friction element of a valve, according to this disclosure;

FIG. 2B is a front view of another example of a friction element of a valve, according to this disclosure;

FIG. 3A illustrates a side view of an apparatus for controlling movement of a wire, according to this disclosure;

FIG. 3B illustrates a side view of another example of an apparatus for controlling movement of a wire, according to this disclosure;

FIG. 3C illustrates a side view of a further example of an apparatus for controlling movement of a wire, according to this disclosure;

FIG. 4 illustrates a side cross-sectional view of a system for delivering an implant, according to this disclosure;

FIG. 5A illustrates a perspective cutaway view and detail view of the system for delivering an implant of FIG. 4, according to this disclosure;

FIG. 5B illustrates a perspective cutaway view of the system for delivering an implant of FIG. 4 with the wire pulled slightly further proximally than depicted in FIG. 5A, according to this disclosure;

FIG. 5C illustrates a perspective cutaway view of the system for delivering an implant of FIG. 4 with the wire pulled slightly further proximally than depicted in FIG. 5B, according to this disclosure;

FIG. 5D illustrates a perspective cutaway view of the system for delivering an implant of FIG. 4 with the implant deployed, according to this disclosure;

FIG. 6 illustrates the delivery of an implant into an aneurysm by a system for delivering an implant, according to this disclosure;

FIG. 7A is a flow diagram illustrating a method of deploying an implant, according to aspects of the present disclosure; and

FIG. 7B is a flow diagram illustrating a method of deploying an implant, according to aspects of the present disclosure.

### DETAILED DESCRIPTION

The following detailed description is merely exemplary in nature and is not intended to limit the disclosure or the application and uses of the disclosure. Although the description of the disclosure is in many cases in the context of treatment of intracranial aneurysm, the disclosure may also be used in other body passageways as previously described.

The terms “distal” or “proximal” are used in the following description with respect to a position or direction relative to the treating physician. “Distal” or “distally” are a position distant from or in a direction away from the physician. “Proximal” or “proximally” or “proximate” are a position near or in a direction toward the physician.

As discussed herein, a “patient” or “subject” can be a human or any animal. It should be appreciated that an animal can be a variety of any applicable type, including, but not limited to, mammal, veterinarian animal, livestock animal or pet-type animal, etc. As an example, the animal can be a laboratory animal specifically selected to have certain characteristics similar to a human e.g., rat, dog, pig, monkey, or the like.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±20% of the recited value, e.g., “about 90%” may refer to the range of values from 71% to 99%.

When used herein, the terms “tubular” and “tube” are to be construed broadly and are not limited to a structure that is a right cylinder or strictly circumferential in cross-section or of a uniform cross-section throughout its length. For example, the tubular structure or system is generally illustrated as a substantially right cylindrical structure. However, the tubular system may have a tapered or curved outer surface without departing from the scope of the present invention.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It must also be noted that, as used in the specification and the appended claims, the singular forms “a.” “an,” and “the” include plural referents unless the context clearly dictates otherwise. Ranges can be expressed herein as from “about” or “approximately” one particular value and/or to “about” or “approximately” another particular value. When such a range is expressed, other exemplary examples include from the one particular value and/or to the other particular value.

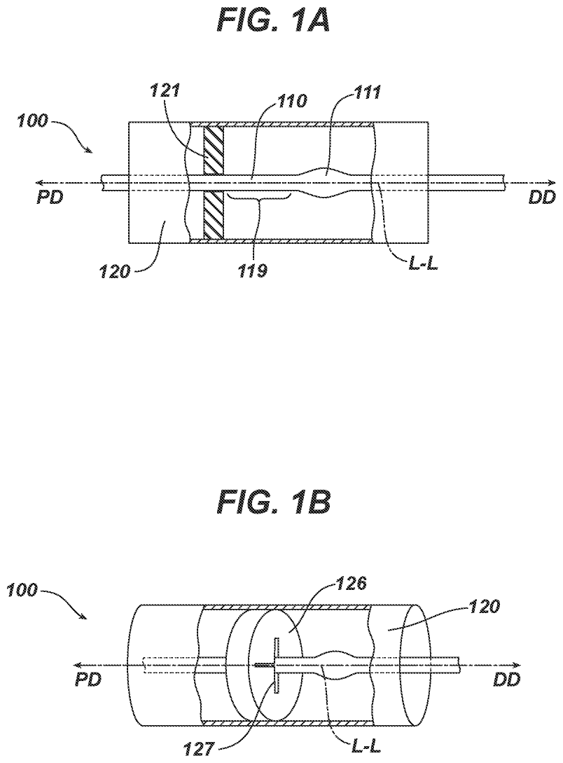

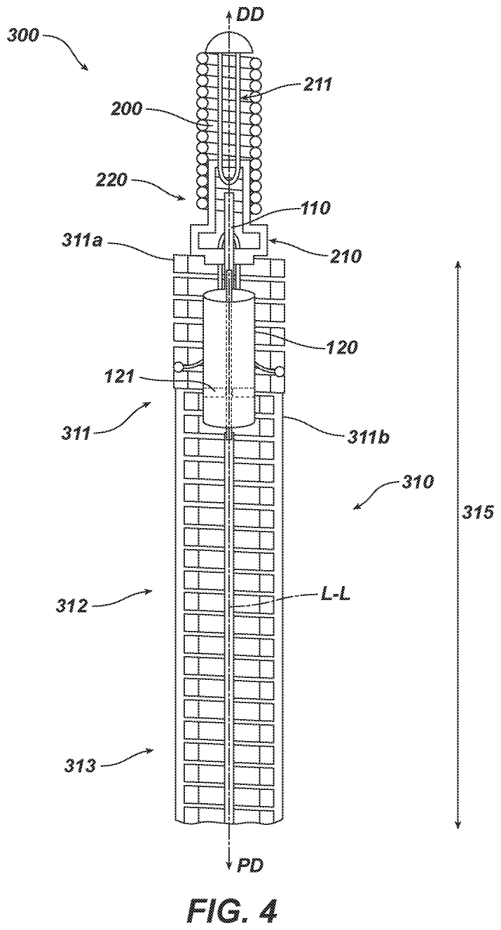

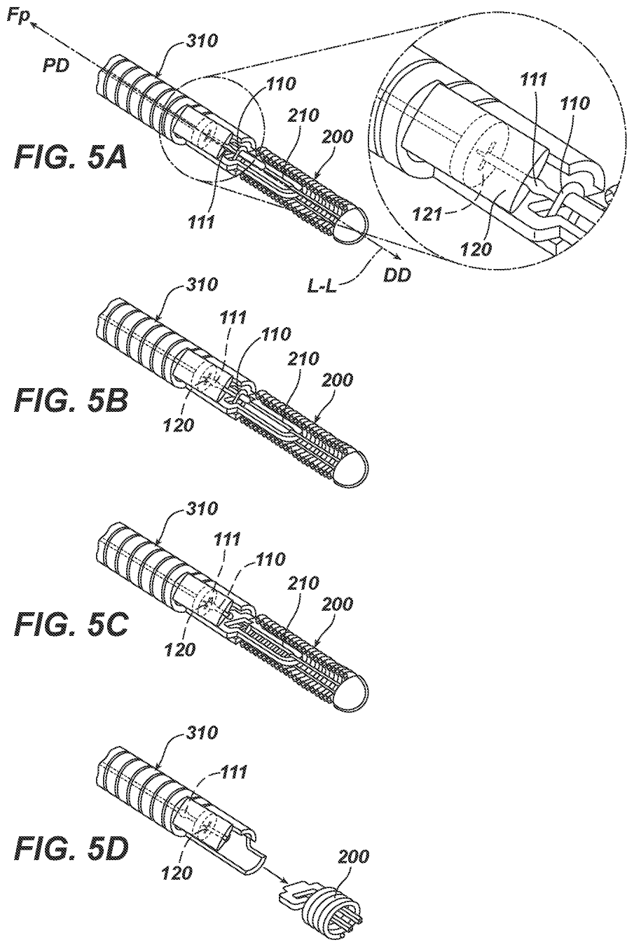

Reference is now made to FIG. 1A-1B which show an apparatus 100 controlling movement of a wire 110 configured to deploy an implant 200 (shown in more detail in FIGS. 4-6). The apparatus 100 can include a wire 110 having a bump 111 and a valve 120. The valve 120 can include a friction element 121 configured to apply a friction force to the wire 110. The wire 110 can be configured to deploy an implant 200 for example, as shown in FIGS. 5A-5D, upon a proximal retraction of the wire 110.

As shown in FIG. 1B, in some examples, the friction element 121 can include a flexible body 126. The flexible body 126 can include a lumen 127 disposed along a longitudinal axis L-L of the valve 120, the lumen 127 being sized such that the bump 111 engages the friction element 121 while the rest of the wire 110 does not engage the friction element 121. Until the bump 111 moves proximal of the friction element 121, the implant 200 cannot be deployed.

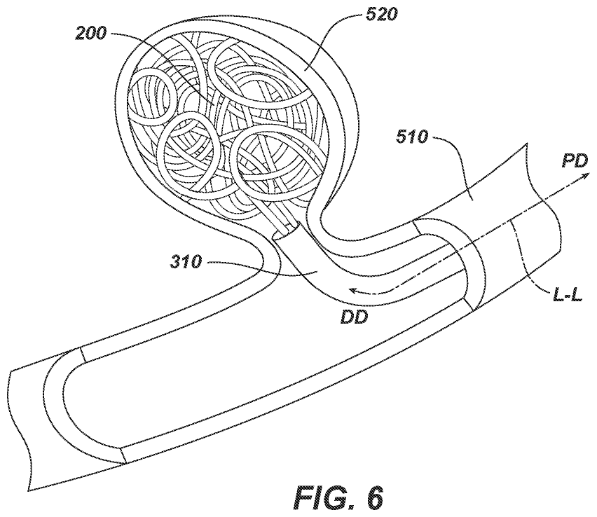

The friction element 121 and bump 111 are sized relative to each other such that unless a deliberate proximal pull force Fp (see FIG. 5A) is applied, the bump 111 cannot pass proximal of the friction element 121. The pull force Fp can be sufficient to overcome the friction element 121 and deploy the implant 200. As illustrated in FIG. 1A, there can be a gap 119 between the bump 111 and the friction element 121. This gap 119 can act as “play” in the pull wire 110, so that as the implant 200 is traversed through the vasculature 510 to the aneurism 520 in the delivery tube 310 (see FIG. 6) the wire 110 can move proximally but cannot overcome the friction element 121. As noted above, friction between the delivery tube 310 and the wire 110 can be generated as the implant 200 is being delivered, however, the force generated does not meet or exceed the pull force Fp. Although friction element 121 shown in FIGS. 1A-1B extends through only a portion of the valve, friction element 121 can extend further in the proximal PD and/or distal directions DD in the valve 120. Generally, the further that friction element 121 extends along the longitudinal axis L-L, the more friction it will apply to wire 110.

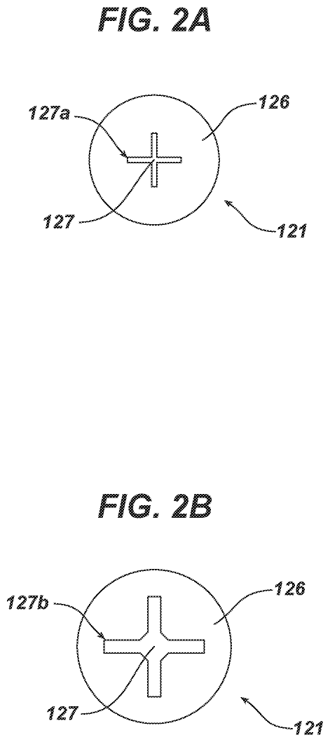

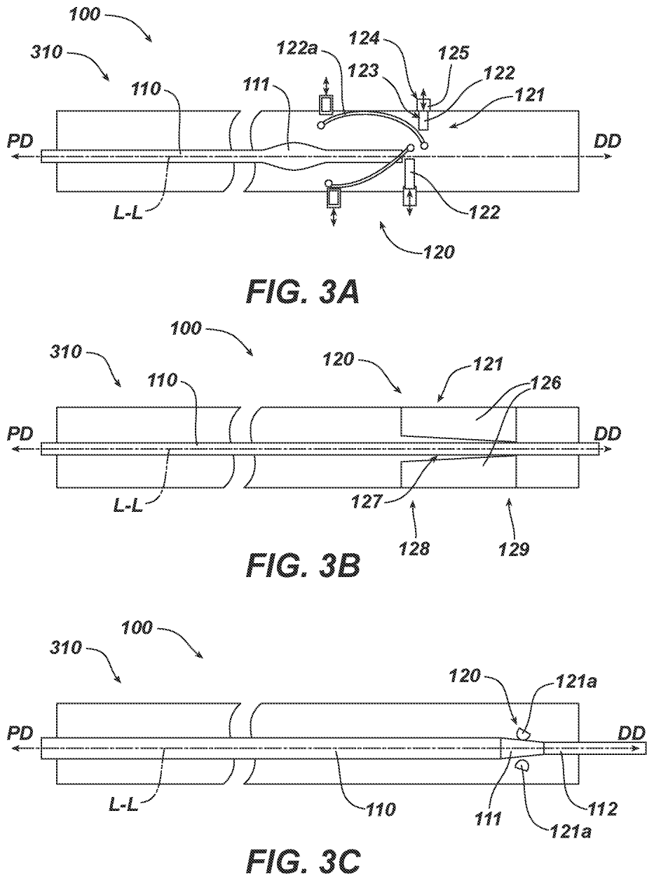

FIGS. 2A-2B show the friction element 121 and flexible body 126 in greater detail. As shown in FIG. 2A, the lumen 127 can be a plus-shaped cross-section 127a. Alternatively, as shown in FIG. 2B, the lumen 127 can be a plus-shaped cross section with an enlarge central portion 127b. As can be seen, the cross-section of lumen 127 depicted in FIG. 2A is smaller than that of the lumen 127 depicted in FIG. 2B. Generally, a larger cross-section of lumen 127 results in a lower pull force Fp. The lumen 127 can be shaped and/or sized such that the wire 110 is in contact with the friction element 121 at all points along the length of wire 110 disposed in lumen 127. Alternatively, lumen 127 can be shaped and/or sized such that the wire 110 is frictionally engaged by the friction element 121 only at bump 111. In some examples, described in more detail in relation to FIG. 3B below, the wire 110 does not include a bump, and friction is applied by friction element 121 to the wire 110 along the length of wire 110 disposed in lumen 127.

The pull force Fp is also related to the compliance of friction element 121—if friction element 121 is made of a lower durometer material, the pull force Fp will be lower compared to if a higher durometer material were used.

Bump 111 can be tapered to facilitate a gradual increase in the resistance to proximally directed pulling force. Furthermore, the further that friction element 121 extends along the longitudinal axis L-L i.e. the longer friction element 121 is, the longer the user will have to apply pull force Fp over a greater distance longitudinally in order to deploy implant 200.

FIG. 3A shows an example of an apparatus 100 for controlling movement of a wire 110. The apparatus can include a wire 110 having a bump 111 and the valve 120 can include a friction element 121 configured to apply a friction force to the wire 110, wherein the friction force varies based on one or more of: a speed at which the wire 110 is pulled through the friction element 121, a direction in which the wire 110 is pulled through the friction element 121, a contact between the bump 111 and the friction element 121, and the shape of lumen 127. The friction element 121 can include a block 122 configured to move radially with respect to a longitudinal axis L-L of the valve 120 from a first position 123 to a second position 124. The block 122, when in the first position 123, hinders the wire 110 from moving longitudinally in the valve 120. The block 122, when in the second position 124, allows the wire 110 to move longitudinally in the valve 120. In some examples, the friction element 121 can further include a piezoelectric element 125 configured to resist a sudden movement of the block 122 from the first position 123 to the second position 124 and to allow a gradual controlled movement of the block 122 from the first position 123 to the second position 124. In some examples, the piezoelectric element 125 can be configured to allow a movement of the block 122 from the first position 123 to the second position 124 when the wire 110 is pulled with a force greater than a minimum deployment force. In some examples, block 122 can include an arcuate intermediate lever arm 122a configured to move radially responsive to impingement with piezoelectric element 125 and wire 110. A portion of the arcuate intermediate lever arm 122a that impinges wire 110 can move radially further than a portion that impinges piezoelectric element 125. Valve 120 can be disposed at least partially in a delivery tube 310.

FIG. 3B shows an example of an apparatus 100 for controlling movement of a wire 110. The apparatus can include a wire 110 configured to deploy an implant 200 upon a proximal retraction of the wire 110 and a valve 120. The valve 120 can include a friction element 121 configured to apply a friction force to the wire 110. The friction element 121 can include a flexible body 126 including a lumen 127 disposed along a longitudinal axis L-L of the valve 120. The lumen 127 can narrow from a proximal end 128 to a distal end 129 such that the wire 110 does not engage the friction element 121 at the proximal end and the wire 110 does engage the friction element 121 at the distal end 129. Valve 120 can be disposed at least partially in a delivery tube 310.

FIG. 3C shows an example of an apparatus 100 for controlling movement of a wire 110. The wire 110 can include a narrowed portion 112 distal to the bump 111. The friction element 121 can include one or more flexible blocks 121a configured to engage the wire 110 proximal to the bump 111 and to not engage the narrowed portion 112 of the wire 110. The valve 120 can be configured to allow the proximal retraction of the wire 110 when the wire 110 is pulled with a force greater than a minimum deployment force. Valve 120 can be disposed at least partially in a delivery tube 310.

In any of the examples disclosed herein, wherein the friction force applied by the friction element 121 can vary based on one or more of: a speed at which the wire 110 is pulled through the friction element 121, a direction in which the wire 110 is pulled through the friction element 121, contact or lack thereof between the bump 111 and the friction element 121, and perforation design of the friction element 121.

Any of the previously described subcomponents of example apparatus 100, especially valve 120 and its constituent components and configurations, can be used in a system 300 for delivering an implant 200 as described below.

In any of the examples disclosed herein, flexible body 126 can be constructed of flexible materials such as biocompatible silicone, thermoplastic elastomers, or other flexible materials as appreciated by those skilled in the pertinent art. Flexible body 126 can include a high friction surface finish.

FIGS. 4-6 show a system 300 for delivering an implant 200. As shown in FIG. 6, the implant 200 can be delivered through vasculature 510 to an aneurism 520, for example in the brain, through delivery tube 310. As shown in FIG. 4, the system 300 can include a delivery tube 310, a wire 110, and a valve 120. The delivery tube 310 can include a distal spring portion 311, an intermediate flexible portion 312, and a proximal portion 313 which can make up a majority of a length 315 of the delivery tube 310. Valve 120 can include a friction element 121 configured to apply a friction force to the wire 110, wherein the friction force varies based on one or more of: a speed at which the wire 110 is pulled through the friction element 121, a direction in which the wire 110 is pulled through the friction element 121, and a contact between the wire 110 and the friction element 121. In some examples, the implant 200 can be an implantable coil 200 disposed proximate a distal end 311a of the distal spring portion 311, wherein the distal spring portion 311 is configured to deploy the implantable coil 200 distally upon the proximal retraction of the wire 110. The wire 110 can be disposed in the delivery tube 310 and can be configured to deploy an implant 200 upon a proximal retraction of the wire as shown in FIGS. 5A-5D.

In some examples, a detachment feature 210 disposed on a proximal end 220 of the implantable coil 200 is configured to receive the wire 110, and the implantable coil 200 is deployed upon the proximal retraction of the wire 110 through the detachment feature 210. The implant 200 can be deployed upon proximal retraction of the wire 110 through a detachment feature 210. The detachment feature 210 can be disposed on a proximal end 220 of the implant 200 and configured to receive the wire 110.

In any of the examples described herein, valve 120 can be disposed in the delivery tube 310 proximate a proximal end 311b of the distal spring portion 311.

Additional details related to the detachment feature 210, wire 110, bump 111, and deployment of embolic coils more generally can be found in U.S. Pat. No. 11,253,265 and U.S. patent application Ser. No. 17/569,632 which are incorporated by reference as if set forth herein in entirety.

Also disclosed herein is a method 400 of deploying an implant. Generally, the method can include positioning step 402 a distal end of a delivery tube while hindering a premature deployment of an implant and deploying step 404 the implant, wherein deploying the implant can include retracting a wire proximally by pulling the wire with a force greater than a minimum deployment force, the premature deployment of the implant being caused by proximal movement of a distal portion of the wire in relation to a delivery tube.

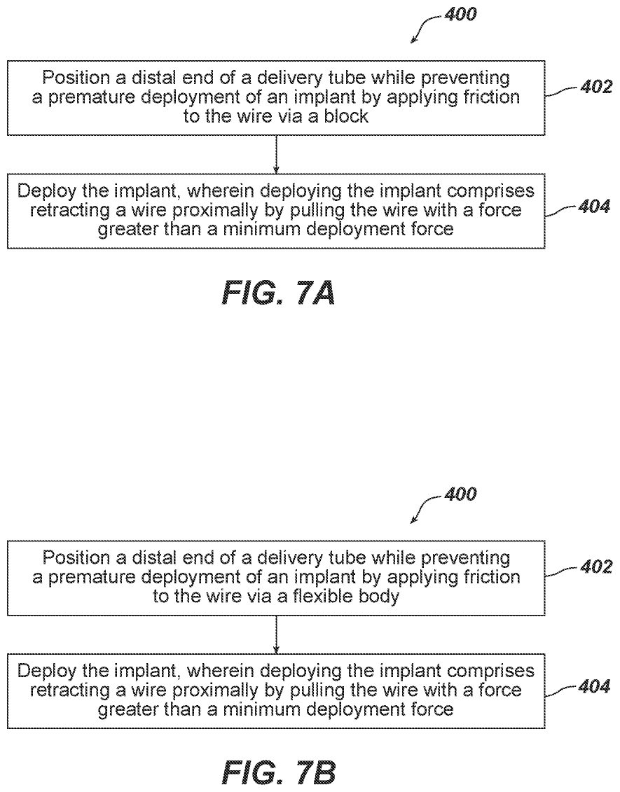

In the example illustrated in FIG. 7A hindering the premature deployment during positioning step 402 can include applying friction to the wire via a block disposed in a valve, the block being configured to move radially with respect to a longitudinal axis L-L of the delivery tube from a first position to a second position. The block, when in the first position, hinders the wire from moving longitudinally in the delivery tube, and, when in the second position, allows the wire to move longitudinally in the valve.

In some examples, applying friction can include providing via a piezoelectric element a resistance to a movement of the block from the first position to the second position, wherein the resistance hinders the movement of the block when the wire is pulled with a force less than the minimum deployment force.

In the example illustrated in FIG. 7B, hindering the premature deployment during positioning step 402 can include applying friction to the wire via a flexible body, wherein the wire passes through a lumen of the flexible body, the lumen configured to allow proximal retraction of the wire when the wire is pulled with a force greater than a minimum deployment force.

As will be appreciated, the method 400 just described can be varied in accordance with the various elements and implementations described herein. That is, methods in accordance with the disclosed technology can include all or some of the steps described above and/or can include additional steps not expressly disclosed above. Further, methods in accordance with the disclosed technology can include some, but not all, of a particular step described above. Further still, various methods described herein can be combined in full or in part. That is, methods in accordance with the disclosed technology can include at least some elements or steps of a first method and at least some elements or steps of a second method.

The disclosed technology described herein can be further understood according to the following clauses:

Clause 1: An apparatus controlling movement of a wire configured to deploy an implant, the apparatus comprising: a wire comprising a bump, wherein the wire is configured to deploy an implant upon a proximal retraction of the wire; and a valve comprising a friction element configured to apply a friction force to the wire, wherein the friction force varies based on one or more of: a speed at which the wire is pulled through the friction element, a direction in which the wire is pulled through the friction element, and a contact between the bump and the friction element.

Clause 2: The apparatus of Clause 1, and wherein the friction element comprises a block configured to move radially with respect to a longitudinal axis of the valve from a first position to a second position, wherein the block when in the first position hinders the wire from moving longitudinally in the valve, and wherein the block when in the second position allows the wire to move longitudinally in the valve.

Clause 3: The apparatus of Clause 2, wherein the friction element further comprises a piezoelectric element configured to resist a sudden movement of the block from the first position to the second position and to allow a gradual movement of the block from the first position to the second position.

Clause 4: The apparatus of Clause 2, wherein the friction element further comprises a piezoelectric element configured to allow a movement of the block from the first position to the second position when the wire is pulled with a force greater than a minimum deployment force.

Clause 5: The apparatus of Clause 1, wherein the friction element comprises a flexible body, the flexible body comprising a lumen disposed along a longitudinal axis in the valve, and wherein the lumen narrows from a proximal end to a distal end such that the bump does not engage the friction element at the proximal end and the bump does engage the friction element at the distal end.

Clause 6: The apparatus of Clause 1, wherein the implant is deployed upon the proximal retraction of the wire through a detachment feature, the detachment feature being disposed on a proximal end of the implant and configured to receive the wire.

Clause 7: The apparatus of Clause 1, wherein the friction element comprises a flexible body, the flexible body comprising a lumen disposed along a longitudinal axis of the valve, wherein the wire comprises a narrowed portion distal to the bump, and wherein the lumen is configured to allow the proximal retraction of the wire when the wire is pulled with a force greater than a minimum deployment force.

Clause 8: A system for delivering an implant, the system comprising: a delivery tube comprising: a distal spring portion; an intermediate flexible portion; and a proximal portion comprising a majority of a length of the delivery tube; a wire disposed in the delivery tube and configured to deploy an implant upon a proximal retraction of the wire; a valve comprising a friction element configured to apply a friction force to the wire, wherein the friction force varies based on one or more of: a speed at which the wire is pulled through the friction element, a direction in which the wire is pulled through the friction element, and a contact between the wire and the friction element.

Clause 9: The system of Clause 8, wherein the valve is disposed in the delivery tube proximate a proximal end of the distal spring portion.

Clause 10: The system of Clause 9, and wherein the friction element comprises a block configured to move radially with respect to a longitudinal axis of the valve from a first position to a second position, wherein the block when in the first position hinders the wire from moving longitudinally in the valve, and wherein the block when in the second position allows the wire to move longitudinally in the valve.

Clause 11: The system of Clause 10, wherein the friction element further comprises a piezoelectric element configured to resist a sudden movement of the block from the first position to the second position and to allow a gradual movement of the block from the first position to the second position.

Clause 12: The system of Clause 10, wherein the friction element further comprises a piezoelectric element configured to allow a movement of the block from the first position to the second position when the wire is pulled with a force greater than a minimum deployment force.

Clause 13: The system of Clause 9, wherein the friction element comprises a flexible body, the flexible body comprising a lumen disposed along a longitudinal axis in the valve, and wherein the lumen narrows from a proximal end to a distal end such that the wire does not engage the friction element at the proximal end and the wire does engage the friction element at the distal end.

Clause 14: The system of Clause 9, wherein the friction element comprises a flexible body, the flexible body comprising a lumen disposed along a longitudinal axis of the valve, and wherein the lumen is configured to allow the proximal retraction of the wire when the wire is pulled with a force greater than a minimum deployment force.

Clause 15: The system of Clause 8, further comprising an implantable coil disposed proximate a distal end of the distal spring portion, wherein the distal spring portion is configured to deploy the implantable coil distally upon the proximal retraction of the wire.

Clause 16: The system of Clause 15, further comprising a detachment feature disposed on a proximal end of the implantable coil and configured to receive the wire, wherein the implantable coil is deployed upon the proximal retraction of the wire through the detachment feature.

Clause 17: A method of deploying an implant, the method comprising: positioning a distal end of a delivery tube while hindering a premature deployment of an implant; and deploying the implant, wherein deploying the implant comprises retracting a wire proximally by pulling the wire with a force greater than a minimum deployment force, the premature deployment of the implant being caused by proximal movement of a distal portion of the wire in relation to a delivery tube.

Clause 18: The method of Clause 17, wherein hindering the premature deployment comprises: applying friction to the wire via a block disposed in a valve, wherein the block is configured to move radially with respect to a longitudinal axis of the delivery tube from a first position to a second position, wherein the block when in the first position hinders the wire from moving longitudinally in the delivery tube, and wherein the block when in the second position allows the wire to move longitudinally in the valve.

Clause 19: The method of Clause 18, wherein applying friction comprises providing via a piezoelectric element a resistance to a movement of the block from the first position to the second position, wherein the resistance hinders the movement of the block when the wire is pulled with a force less than the minimum deployment force.

Clause 20: The method of Clause 17, wherein hindering the premature deployment comprises: applying friction to the wire via a flexible body, wherein the wire passes through a lumen of the flexible body, the lumen configured to allow proximal retraction of the wire when the wire is pulled with a force greater than a minimum deployment force.

Other aspects and features of the present disclosure will become apparent to those of ordinary skill in the art, upon reviewing the detailed description in conjunction with the accompanying figures.

In describing examples, terminology is resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method can be performed in a different order than those described herein without departing from the scope of the disclosed technology. Similarly, it is also to be understood that the mention of one or more components in a device, system, or assembly does not preclude the presence of additional components or intervening components between those components expressly identified.

The descriptions contained herein are examples of the disclosure and are not intended in any way to limit the scope of the disclosure. While particular examples of the present disclosure are described, various modifications to devices and methods can be made without departing from the scope and spirit of the disclosure. For example, while the examples described herein refer to particular components, the disclosure includes other examples utilizing various combinations of components to achieve a described functionality, utilizing alternative materials to achieve a described functionality, combining components from the various examples, combining components from the various example with known components, etc. The disclosure contemplates substitutions of component parts illustrated herein with other well-known and commercially available products. To those having ordinary skill in the art to which this disclosure relates, these modifications are often apparent and are intended to be within the scope of the claims which follow.

What is claimed is:

1. An apparatus controlling movement of a wire configured to deploy an implant, the apparatus comprising:
a wire comprising a bump, wherein the wire is configured to deploy an implant upon a proximal retraction of the wire; and
a valve comprising a friction element configured to apply a friction force to the wire, wherein the friction force varies based on one or more of: a speed at which the wire is pulled through the friction element, a direction in which the wire is pulled through the friction element, and a contact between the bump and the friction element.

2. The apparatus of claim 1, and wherein the friction element comprises a block configured to move radially with respect to a longitudinal axis of the valve from a first position to a second position, wherein the block when in the first position hinders the wire from moving longitudinally in the valve, and wherein the block when in the second position allows the wire to move longitudinally in the valve.

3. The apparatus of claim 2, wherein the friction element further comprises a piezoelectric element configured to resist a sudden movement of the block from the first position to the second position and to allow a gradual movement of the block from the first position to the second position.

4. The apparatus of claim 2, wherein the friction element further comprises a piezoelectric element configured to allow a movement of the block from the first position to the second position when the wire is pulled with a force greater than a minimum deployment force.

5. The apparatus of claim 1, wherein the friction element comprises a flexible body, the flexible body comprising a lumen disposed along a longitudinal axis in the valve, and wherein the lumen narrows from a proximal end to a distal end such that the bump does not engage the friction element at the proximal end and the bump does engage the friction element at the distal end.

6. The apparatus of claim 1, wherein the implant is deployed upon the proximal retraction of the wire through a detachment feature, the detachment feature being disposed on a proximal end of the implant and configured to receive the wire.

7. The apparatus of claim 1, wherein the friction element comprises a flexible body, the flexible body comprising a lumen disposed along a longitudinal axis of the valve, wherein the wire comprises a narrowed portion distal to the bump, and wherein the lumen is configured to allow the proximal retraction of the wire when the wire is pulled with a force greater than a minimum deployment force.

8. A system for delivering an implant, the system comprising:
a delivery tube comprising:
a distal spring portion;
an intermediate flexible portion; and
a proximal portion comprising a majority of a length of the delivery tube;
a wire disposed in the delivery tube and configured to deploy an implant upon a proximal retraction of the wire;
a valve comprising a friction element configured to apply a friction force to the wire, wherein the friction force varies based on one or more of: a speed at which the wire is pulled through the friction element, a direction in which the wire is pulled through the friction element, and a contact between the wire and the friction element.

9. The system of claim 8, wherein the valve is disposed in the delivery tube proximate a proximal end of the distal spring portion.

10. The system of claim 9, and wherein the friction element comprises a block configured to move radially with respect to a longitudinal axis of the valve from a first position to a second position, wherein the block when in the first position hinders the wire from moving longitudinally in the valve, and wherein the block when in the second position allows the wire to move longitudinally in the valve.

11. The system of claim 10, wherein the friction element further comprises a piezoelectric element configured to resist a sudden movement of the block from the first position to the second position and to allow a gradual movement of the block from the first position to the second position.

12. The system of claim 10, wherein the friction element further comprises a piezoelectric element configured to allow a movement of the block from the first position to the second position when the wire is pulled with a force greater than a minimum deployment force.

13. The system of claim 9, wherein the friction element comprises a flexible body (126), the flexible body comprising a lumen disposed along a longitudinal axis in the valve, and wherein the lumen narrows from a proximal end to a distal end such that the wire does not engage the friction element at the proximal end and the wire does engage the friction element at the distal end.

14. The system of claim 9, wherein the friction element comprises a flexible body, the flexible body comprising a lumen disposed along a longitudinal axis of the valve, and wherein the lumen is configured to allow the proximal retraction of the wire when the wire is pulled with a force greater than a minimum deployment force.

15. The system of claim 8, further comprising an implantable coil disposed proximate a distal end of the distal spring portion, wherein the distal spring portion is configured to deploy the implantable coil distally upon the proximal retraction of the wire.

16. The system of claim 15, further comprising a detachment feature disposed on a proximal end of the implantable coil and configured to receive the wire, wherein the implantable coil is deployed upon the proximal retraction of the wire through the detachment feature.

17. A method of deploying an implant, the method comprising:
positioning a distal end of a delivery tube while hindering a premature deployment of an implant; and
deploying the implant, wherein deploying the implant comprises retracting a wire proximally by pulling the wire with a force greater than a minimum deployment force, the premature deployment of the implant being caused by proximal movement of a distal portion of the wire in relation to a delivery tube.

18. The method of claim 17, wherein hindering the premature deployment comprises:
applying friction to the wire via a block disposed in a valve, wherein
the block is configured to move radially with respect to a longitudinal axis of the delivery tube from a first position to a second position, wherein
the block when in the first position hinders the wire from moving longitudinally in the delivery tube, and wherein
the block when in the second position allows the wire to move longitudinally in the valve.

19. The method of claim 18, wherein applying friction comprises providing via a piezoelectric element a resistance to a movement of the block from the first position to the second position, wherein the resistance hinders the movement of the block when the wire is pulled with a force less than the minimum deployment force.

20. The method of claim 17, wherein hindering the premature deployment comprises:
applying friction to the wire via a flexible body, wherein the wire passes through a lumen of the flexible body,
preventing premature deployment by allowing limited proximal retraction of the wire based on the applied friction; and
allowing unlimited proximal retraction of the wire when the wire is pulled with a force greater than a minimum deployment force to deploy the implant.
